(19) United States
(12) Patent Application Publication
(10) Pub. No.: US 2009/0175970 A1
(43) Pub. Date: Jul. 9, 2009

(54) Stabilized Plant Extract and Its Therapeutic Use

(75) Inventor: Klaus Diergarten, Herrsching (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: VERITRON LIMITED, London (GB)

(21) Appl. No.: 12/402,964

(22) Filed: Mar. 12, 2009

Related U.S. Application Data
(63) Continuation of application No. 11/814,651.

(30) Foreign Application Priority Data
Jan. 26, 2005 (GB) 0501654.8

(51) Int. Cl. A61K 36/28; A01H 5/00; C08F 226/10; A61P 35/00

(52) U.S. Cl. 424/764; 800/298; 526/258

(57) ABSTRACT

A plant extract, obtainable by aqueous extraction, is stabilised by the presence of a water-soluble agent capable of chelating the extract or a component thereof.

### FIELD OF THE INVENTION

This invention relates to a stabilised plant extract and its therapeutic use.

### BACKGROUND OF THE INVENTION

The therapeutic effect of herbs and other plants has been acknowledged for millennia. There has however been little rational analysis of such effects. This is despite the fact that natural products are of increasing interest, not least because of the possibility that they may have specific therapeutic effects, without major side effects.

Before the useful therapeutic effects of plants can be properly analysed and utilised under today's strict regulatory requirements, it is desirable that they should be obtainable under reproducible conditions, and in stabilised form. While various plant extracts are available without regulation, it is clear that the most beneficial properties cannot be realised without proper formulation and regulation.

A composition having remarkable therapeutic effects, including the treatment of tumours, is disclosed in WO03/101479. One of the components of the most preferred embodiment is a camomile extract which is described as an anti-irritant. No pharmaceutical property is ascribed to the extract. It is suggested that the aqueous extract (for which a particular extraction procedure is given) has a palliative effect when administered by the preferred route, i.e. injection.

### SUMMARY OF THE INVENTION

The present invention is based on the surprising discovery than an aqueous extract of the type described in WO03/101479 can be effectively stabilised by the presence of polyvinylpyrrolidone (PVP), and the inventor believes that other water-soluble chelating agents will also be effective for the same purpose. Thus, an aqueous composition according to the present invention may comprise a plant extract, obtainable by aqueous extraction, and such a water-soluble agent.

As described below, a product of the invention may have utility in a new process for the preparation of the composition described in WO03/101479.

### DESCRIPTION OF PREFERRED EMBODIMENTS

Plants that may be extracted for use in the invention include Matricaria Chamomile, Acacia Senegal, Nigella Sativa, Calendula Officinalis L. (Compositae), Achillea Millefolium L. (Compositae) and Youngia Japonica L. The extract may be obtained from any part of the plant, e.g. the stem, leaves or flowers. An aqueous extract is preferred.

The available evidence suggests that an extract of such herbal substances obtained as described below may cause an increase in the level of the cytokine IL-6. This is a good measure of the desirable properties of this extract. Other cytokines that may be affected, in order to obtain the desirable properties of the invention, are other interleukins such as IL-7, IL-8, IL-9 and IL-10, and also tumour necrosis factor (TNF).

The extract may be obtained by any suitable procedure, including methods known to those of ordinary skill in the art. The extract may be obtained by using an aqueous or organic medium, and separated from other components by filtration, chromatography etc. For example, a material that may be used in the invention is derived from the dried flowerheads of the composite plant Matricaria chamomilla, the seeds of the plant Nigella Sativa, the gum of the plant Acacia Senegal, or one or more materials therein, include polysaccharides, glycoproteins, volatile oils, azolene, anthemic acid, apogenin, glycosides and other substances.

The preferred water-soluble chelating agent is PVP. Other such agents may be suitable for use in the invention. The amount of the chelating agent that can be used will be dependent on factors such as the component to be stabilised, other components and the intended use. A suitable amount can readily be determined by one skilled in the art.

An aqueous composition may comprise one or more components as described in WO03/101479, the content of which is incorporated herein by reference. One such component for use in the invention is an antioxidant. The function of this component may be to prevent the formation of S—S bridges by oxidation of cysteine residues. Disulfide linkages are caused by many oxidising agents, and cause loss of enzymatic activity. Alternatively or in addition, the antioxidant may inhibit the production of oxygen radicals (free radicals) as a by-product of the normal metabolism of oxygen. These oxygen radicals are very damaging to cell membranes, proteins, lipids and DNA. Oxidative damage accumulates with age and is considered to be a major contributor to ageing and the development of degenerative diseases (e.g. cancer, cardiovascular disease, immune system decline, etc).

Suitable antioxidants for use in the invention are small molecules such as vitamin C, A and E. It will be appreciated that a suitable precursor of any such compound may be used, e.g. β-carotene. The preferred antioxidant for use in this invention is vitamin C, e.g. as Sodium ascorbate or ascorbic acid. A suitable dosage of this component is 1 to 500 mg/kg/day.

Alternatively or in addition, this component may comprise one or more components of vitamin B. Many enzymes catalyse reaction of their substrates only in the presence of a specific non-protein molecule, i.e. a coenzyme. Coenzymes frequently contain B vitamins as part of their structure. One or more of vitamins B1 (thiamine hydrochloride), B2 (riboflavin sodium phosphate), PP (nicotinamide), B6 (pyridoxine hydrochloride) and B5 (dexpanthenol) may be used. The amount of each such component is, for example, 0.1 to 50 mg/kg/day.

A second component for use according to the present invention is a metal salt that provides metal ions, in vivo. The nature of the anion is not critical, and will generally be chosen to be non-toxic and of suitable solubility or other appropriate compatibility with other components of the medicament. Many metal ions act as positive modifiers, and certain enzymes require the presence of metal ion for full activity. The function of the metal ion may be to complement the coenzyme. The ion may be, for example, Na, K or multivalent such as Fe, Mo, Mg, Mn, Ca, Zn, Cu or Co. This may be in the form of a salt, of which many examples are known, e.g. with any inorganic acid such as HCl or H₂SO₄, or an organic acid such as acetic, ascorbic, citric, gluconic, glutamic, maleic, malic or succinic acid. A preferred component of this type is Calcium Gluceptate. A typical dosage of this component is 1 to 1000 mg/kg/day.

A third component for use according to the invention may be an agent that increases the permeability of cell membranes or otherwise enhances transport, e.g. by action on receptors. This material may enhance the ability of the cells that need treatment to receive the other active material or materials that may be included in the novel medicament, especially in human therapy (it may not be required in veterinary medicine). A preferred agent of this type is insulin or a growth hormone. A typical dosage of insulin is 1 to 1000 IU/kg/day.

Another component is an antihistamine. Such a material can not only prevent or reduce abnormal reactions, especially allergic reaction, but also prevent the accumulation of substances which block transport, e.g. by binding to cell membrane receptors.

As is known to those of ordinary skill in the art, antihistamines work by competing with histamine released by mast cells and basophils for histamine receptors on the mucosa of the eyes, nose, bronchial airways and skin. The antihistamine binds to the receptor and prevents histamine attachment, thereby blocking the effect of histamine in the tissues. Antihistamine drugs counteract the physiological effects of histamine production, in allergic reactions and colds.

Antihistamines can be divided into classical and non-sedating antihistamines. There are many examples of such compounds, including acrivastine, azatadine, azelastine, brompheniramine, carbinoxamine, cetirizine, chlorpheniramine, clemastine fumarate, cyproheptadine, diphenhydramine, doxepin, hydroxyzine, fexofenadine, loratadine, meclizine, phenindamine, promethazine, pyrilamine and tipolidine.

A preferred material for use in the invention is chlorpheniramine maleate. A suitable dosage of such a component is 0.1 to 50 mg/kg/day.

It is also believed that certain types of honey may comprise components that provide an activity that is analogous to that obtained from the components of the plants described above.

Other active materials may also be given to the subject. Although it is not believed that further materials are necessary, it has been found that certain steroids and vitamins, typically given orally, can support or enhance the effect of the medicament. Suitable steroid hormones may increase the synthesis of specific proteins, by unmasking certain cistrons, with the assistance of essential metabolites such as vitamins and amino acids. Examples of suitable steroids are estradiol, nandrolone and estriol. Vitamins such as A, D and/or E may also be given. The function of vitamin A may be to preserve the integrity of epithelial tissue, to play a role in protein synthesis, and to stabilise cell membranes and also subcellular membranes.

As is conventional in medicine, a medicament for use in the invention may comprise other components, depending on the intended effect, the nature of the formulation, the route of administration, and other factors that are known to those skilled in the art. Thus, for example, the medicament may be formulated in water, e.g. to provide an aqueous solution or suspension suitable for injection. It may be desirable to include in any such formulation one or more additional substances that aid dissolution or suspension of active components, such as an organic or a polar solvent. The composition may comprise conventional excipients, for example, phenol (which acts as a preservative).

Compositions for use according to the invention can be formulated by methods known to those skilled in the art. Pharmaceutically acceptable components should be used. The term “pharmaceutically acceptable” refers to those properties and/or substances which are acceptable to the patient from a pharmacological/toxicological point of view and to the manufacturing pharmaceutical chemist from a physical/chemical point of view regarding factors such as formulation, stability, patient acceptance and bioavailability.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients such as, for example, inert diluents such as calcium carbonate, sodium carbonate, lactose, calcium phosphate or sodium phosphate; granulating and disintegrating agents, for example corn starch or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated, to form osmotic therapeutic tablets for control release.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

The composition may be an aqueous solution or suspension. It may contain the active materials in admixture with suitable excipients. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents, for example a naturally occurring phosphatide such as lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol, such as a polyoxyethylene with partial esters derived from fatty acids and hexitol anhydrides, for example polyoxyethylene sorbitan monooleate. Aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents (such as those set forth above) and flavouring agents may be added to provide a palatable oral preparation. These compositions may be preserved by the addition of an anti-oxidant such as Ascorbic Acid or Sodium ascorbate or ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified above. Sweetening, flavouring and colouring agents may also be present.

A pharmaceutical composition for use in the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated using suitable dispersing or wetting agents and suspending agents, examples of which have been mentioned above. A sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition may also be administered in the form of suppositories for rectal administration of the drug. Such compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, suitable compositions are in the form of, for example, creams, ointments, jellies, solutions or suspensions. For the purposes of this specification, topical application includes mouth washes and gargles.

As indicated above, composition of the invention may be given by injection. Intramuscular injection is preferred, although any parenteral administration is suitable.

It may also be preferred that the composition is given orally, although insulin should not usually be included in an oral formulation. Oral administration may be particularly preferred for veterinary medicine.

Although some indication has been given as to suitable dosages of certain materials, the exact dosage and frequency of administration depend on several factors. These factors include the particular components that are used, the particular condition being treated, the severity of the condition, the age, weight and general physical condition of the particular patient, and other medication the individual may be taking, as is well known to those skilled in the art.

A composition of the invention may be prepared simply by mixing the components, preferably using water for injection. Such a preparation is described in the following Example (which illustrates the invention).

### Example

The following were formulated (using camomile extract as described in WO03/101479):

| | |
|---|---|
| Filtered, Preserved Camomile Extract | 1849.88 g |
| Insulin (Actrapid HM) | 98.43 g |
| Kollidon Solution | 264.71 g |
| Chorpheniramine Maleate | 3.28 g |
| Dexpanthenol (Vitamin B5) | 1.97 g |
| Nicotinamide (Vitamin PP) | 13.11 g |
| Pyridoxine-HCl (Vitamin B6) | 1.31 g |
| Riboflavine Na Phosphate (Vitamin B2) | 1.79 g |
| Thiamine Chloride HCl (Vitamin B1) | 3.28 g |
| Kollidon solution | 211.17 g |
| Water for injection | 71.08 g |

1. A Kollidon solution was prepared by dissolving 213.3 g Kollidon 17 PF in 266.6 g water for injection, with stirring, until a clear solution was obtained. 2.67 g 0.5 NaOH was added, with stirring, to a pH of between 7.0 and 7.4. A clear, colourless solution was obtained.
2. A vitamin mixture was prepared by dissolving the Dexpanthenol, Nicotinamide, Pyridoxine HCl, Rivoflavine Na Phosphate, Thiamine Cl-HCl and Chlorpheniramine Maleate in 71.08 g water for injection, with stirring, for 20 minutes. 211.17 g of the Kollidon solution was added with stirring, for 10 minutes. A clear yellow-orange solution was obtained. The vessel was closed.
3. 264.71 g of the Kollidon solution was added slowly, with stirring, to 98.43 g insulin, over 10 minutes. A clear, colourless solution was obtained. The vessel was closed.
4. The filtered, preserved camomile extract (FCE), stored for 1 week at 2-8° C., was allowed to warm to room temperature. A clear brown solution, pH 5.5-6.5, was obtained.
5. The insulin mixture (3) was added slowly, with stirring, over 10 minutes, to the vitamin mixture (2). A clear, yellow solution was obtained, to which the FCE (4) was added slowly with stirring, over 10 minutes. A clear, yellow-brown solution, pH 4.5-6.5, was obtained. This solution was passed through a sterile 0.2 μm filter. 4 ml of this solution was put in a 5 ml sterile glass vial and sealed with a rubber stopper and flanged cap.

1. A plant extract, obtainable by aqueous extraction, stabilized by the presence of a water-soluble agent capable of chelating the extract or a component thereof.

2. An extract according to claim 1, wherein the plant is an herb.

3. An extract according to claim 2, wherein the herb is chamomile.

4. An extract according to claim 1, comprising one or more components of Nigella Sativa.

5. An extract according to claim 1, comprising one or more components of Acacia Senegal.

6. An extract according to claim 1, wherein the agent is PVP.

7. A plant extract according to claim 1, for therapeutic use.

8. A composition according to claim 7, additionally comprising one or more components of a formulation as described in WO03/101479.

9. A composition according to claim 8, which comprises vitamin C.

10. A composition according to claim 8, which comprises Ca ions.

11. A composition according to claim 9, which comprises Ca ions.

12. An extract according to claim 2, wherein the agent is PVP.

13. An extract according to claim 3, wherein the agent is PVP.

14. An extract according to claim 4, wherein the agent is PVP.

15. An extract according to claim 5, wherein the agent is PVP.

16. A plant extract according to claim 12, for therapeutic use.

17. A plant extract according to claim 13, for therapeutic use.

18. A plant extract according to claim 14, for therapeutic use.

19. A plant extract according to claim 15, for therapeutic use.
